(19) United States
(12) Patent Application Publication
(10) Pub. No.: US 2009/0197300 A1
(43) Pub. Date: Aug. 6, 2009

(54) MUTUALLY EXCLUSIVE DOMAIN FOLDING MOLECULAR SWITCH AND METHOD OF SYNTHESIS THEREOF

(76) Inventors: James S. Butler, Arlington, MA (US); Stewart N. Loh, Manlius, NY (US); Jeung-Hoi Ha, Manlius, NY (US); Tracy L. Radley, Liverpool, NY (US)

Correspondence Address: SANDER RABIN MD JD;A Professional Corporation, Saratoga Technology Accelerator, 125 High Rock Avenue, Saratoga Springs, NY, 12866, US

(21) Appl. No.: 11/670,966

(22) Filed: Feb. 3, 2007

Related U.S. Application Data
(63) Continuation-in-part of application No. 10/802,516, filed on Mar. 17, 2004.

(51) Int. Cl. C12P 21/00; C12N 9/96

(52) U.S. Cl. 435/69.1; 435/188

(57) ABSTRACT

The invention is a fusion protein, embodying a mutually exclusive folding domain molecular switch, wherein the free energy released by folding of a first domain of the fusion protein drives an unfolding of a second domain of the fusion protein, and vice versa. The fusion protein is engineered so that folding the first domain unfolds the second domain, and vice versa, making the folded and unfolded states of the domains mutually exclusive. This is accomplished by insertion of an insert protein GCN4 into a surface loop of a target, protein barnase, subject to die topological design, criterion that the N—C terminal length of GCN4 be at least two-times greater than the Cα-Cα length of a surface loop of barnase. In the absence of the ligand AP-1, barnase is more stable and is folded and active. The presence of AP-1 induces folding of GCN4, forcibly unfolding and inactivating barnase.

### PARENT CASE TEXT

This application is a Continuation-in-Part of U.S. patent application Ser. No. 10/802,516, filed Mar. 17, 2004, the entirety of which is incorporated herein by reference.

### FEDERAL GRANT

Some of the research described in this application was funded by Grant R01 GM069755 from the National Institutes of Health. The U.S. government may therefore have certain rights in the invention.

### 1.0 BACKGROUND OF THE INVENTION

### 1.1 Technical Field

The invention relates generally to a fusion protein that functions as molecular switch to modulate the bioactivity of other proteins.

### 1.2. RELATED ART

### 1.2.1 Referenced Publications

All references cited herein, including journal articles or abstracts, published or corresponding U.S. or foreign patent applications, issued U.S. or foreign patents, or any other references, are entirely incorporated by reference herein, to disclose and describe the methods and/or materials in connection with which the publications or documents are cited, including all data, tables, figures, and text presented in the cited references. Additionally, the entire contents of the references cited within the references cited herein are also entirely incorporated by references.

Citation of any references herein is not intended as an admission that the references is pertinent prior art, or considered material to the patentability of any claim of the present application. Any statement as to content or a date of any references is based on the information available to applicant at the time of filing and does not constitute an admission as to the correctness of such a statement. The dates of publication provided may be different from the actual publication dates which may need to be independently confirmed.

### 1.2.2 Parent Application

As disclosed in Parent U.S. patent application Ser. No. 10/802,516, filed Mar. 17, 2004, (10/802,516 Application) some of the inventors herein synthesized a novel two-domain fusion protein, comprising an insert protein and a target protein, wherein the mechanical stress imposed by the folded structure of the insert protein forces the target protein to unfold, and vice versa. The fusion protein disclosed in the 10/802,516 Application, functions as a mutually exclusive folding domain (“MEFD”) molecular switch.

As described in the 10/802,516 Application, the MEFD switching mechanism is embodied in a fusion protein created by inserting an insert protein into a surface loop of a target protein, subject to the novel and explicitly defined topological design criterion that the linear (straight-line) distance between the amino and carboxyl ends of insert protein (“N—C terminal length”) be at least two times greater than the distance between the termini of the surface loop (“Cα-Gα length”) of the target protein. If the insert protein is more stable than the target protein, the insert protein forcibly stretches and unfolds the target protein. If the target protein is more stable than the insert protein, the target protein stretches and unfolds insert protein.

The fusion protein thus exists in a state of conformational equilibrium in a thermodynamic tug-of-war wherein only one protein domain can exist in its folded state at any given time. In the 10/802,516 Application, the insert protein was human ubiquitin (“U”) and the target protein was the bacterial ribonuclease barnase (“Bn”). The resultant Bn-U fusion protein (“BU”) exists in a conformational equilibrium that is reversible, cooperative, and controllable by external factors such as temperature, the presence of absence of a denaturant, and ligand binding.

Ribonucleases, such as Bn, are hydrolase enzymes that break linkages between nucleotides in ribonucleic acid. They are accordingly highly cytotoxic. A major problem with their use as therapeutic agents, such as, for example, as pharmacologic agents in the treatment of cancer, is that their cytotoxicity is indiscriminate. Currently available ribonuclease pharmacologic agents kill normal as well as neoplastic cells, and the side effects of their use can be severe. Additionally, currently available ribonuclease agents demonstrate poor bioavailability owing to their rapid degradation by the liver and their difficulty in passing through both normal and neoplastic cell membranes.

By means of the molecular switching demonstrated by the BU, as disclosed in the 10/802,516 Application, the catalytic activity of Bn was made controllable for the first time.

### 2.0 SUMMARY OF THE INVENTION

The present invention is a novel fusion protein that also embodies the MEFD molecular switching mechanism disclosed in the 10/802,516 Application, The fusion protein comprises an insert protein, such as the ligand-binding polypeptide GCN4, (“GCN4”) having an insert (regulatory or binding) domain lying between an amino terminal and a carboxyl terminal of the insert protein, the insert domain being associated with a first quantity of free energy; and, a target protein, such as barnase (“Bn”) having a surface loop that begins at an alpha carbon of a first amino acid of the surface loop and terminates at an alpha carbon of a second amino acid of the surface loop, the surface loop comprising a target (catalytic or cytotoxic) domain of the target protein, the target domain being associated, with a second quantity of free energy, wherein, the insert protein is inserted within the surface loop between the alpha carbon of the first amino acid of the surface loop and the alpha carbon of the second amino acid of the surface loop, such that an N—C length (about 75 Å) of the insert protein is at least two-times greater than a Cα-Cα length (about 10 Å) of the surface loop of the target protein.

The insert domain exists in either a folded or unfolded conformation and the target domain exists in either a folded or unfolded conformation. The insert domain and the target domain comprise a cooperative and reversible conformational equilibrium such that if the insert domain is in its folded conformation, the target domain is in its unfolded conformation and vice versa. The insert domain and the target domain are disenabled from simultaneously co-existing in their respective folded conformations; and the insert domain and the target domain are disenabled from simultaneously co-existing in their respective unfolded conformations.

The cooperative and reversible conformational equilibrium may be determined by a controllable effector signal, for example, a ligand such as the APT consensus DNA oligonucleotide.

Any excess of die first quantity of free energy of the insert domain that is not necessary to stabilize the insert domain in its folded conformation is spontaneously transferred, through the structure of said fusion protein, to the target domain to unfold it from its folded conformation; and, any excess of the second quantity of free energy of the target domain that is not necessary to stabilize the target domain in its folded conformation is spontaneously transferred, through the structure of said fusion protein, to the insert domain to unfold it from its folded conformation.

In the novel fusion protein, all or part of the first quantity of free energy is made available to drive a folding of the target domain from its unfolded conformation by means of a controllable effector signal, for example a ligand such as the AP-1 consensus DNA oligonucleotide.

Alternatively summarized, the novel fusion protein is a Barnase-GCN4 fusion protein (“BG”) comprising an insert protein, the ligand-binding polypeptide GCN4, (“GCN4”), having an insert domain, fused to a target protein Bn having a target domain, such that the topological design, criterion prevents the constituent proteins GCN4 and Bn from existing simultaneously in their folded states. Their respective domains engage in a thermodynamic tug-of-war in which the more stable domain forces the less stable domain to unfold. In the absence of the AP-1 consensus DNA oligonucleotide (“AP-1”), Bn is more stable than GCN4, and is therefore folded and active. In the presence of the AP-1, Bn is less stable than GCN4, and is therefore unfolded and inactive. The insert domain of GCN4 is substantially unstructured.

BG binding to APT induces folding of GCN4, forcibly unfolding and inactivating Bn. BG is thus a “natively unfolded” fusion protein that uses ligand binding to AP-1 to switch between partially folded conformations. The characteristic catalytic efficiency of Bn and the characteristic DNA binding affinity and sequence specificity of GCN4 are retained in BG. The conformational equilibrium established between, the insert protein GCN4 and the target protein Bn is specifically reversible and controllable by means of ligand binding to AP-1.

The novel fusion protein BG disclosed herein embodies and provides:
1) a method for assembling fusion proteins with controllable enzymatic activities from a variety of target proteins having catalytic domains and insert proteins having regulatory domains that bind ligands; and,
2) a mechanism wherein the catalytic activity of an enzymatic fusion protein is controlled by ligand binding to a selectable insert protein.

The MEFD molecular switch embodied in BG comprises a molecular mechanism for regulating enzymatic activity. The insert domain of GCN4 in the present invention is inserted into a target domain of Bn as described in the 10/802,516 Application. The resulting BG fusion protein has a new function not present in either constituent protein alone—it senses the presence of a specific DNA sequence, i.e., AP-1; and, the enzymatic activity of Bn is switched on or off depending on whether that DNA sequence is absent or present.

One substantial, specific and credible utility of BG is as a molecular sensor. The substantive nature of this invention arises from the high degree of specificity of the instant fusion protein as a ligand-specific and controllable enzyme. GCN4's insert domain can distinguish the “correct” DNA sequence of the ligand AP-1 from closely related “incorrect” sequences, thereby specifically coupling the activation of the RNA hydrolysis carried out by the target domain of Bn to the presence of the ligand AP-1. RNA hydrolysis is extremely toxic to human cells, bacteria, and RNA viruses. BG can therefore be used to destroy bacteria or viruses, depending on whether the specific GCN4-binding DNA sequence, i.e., the ligand AP-1, is present or absent in that organism.

In laboratory applications, BG has substantial, specific and credible utility as a tool for assaying the presence of a specific DNA sequence (the GCN4 binding sequence) in biological samples. For this utility, RNA hydrolysis is detected by employing a commercially available, colorimetric RNA substrate.

A major goal of biotechnology is the discovery or bioactive proteins and the selective alteration of portions of their amino acid sequences to enhance their stability, that is, to increase the proteins' resistance toward:

1) degradation by human proteases; or,

2) denaturation by, e.g.:
a) heat;
b) detergents;
c) chemicals; and,
d) pH.

Enhancing protein stability is vital to biological applications, such as, for example, when the protein is used as a diagnostic or therapeutic agent), or when, for example, the protein is synthesized in a large-scale industrial processes.

Existing methods for discovering ultra-stable proteins employ high-throughput screens of libraries of protein variants generated randomly in a laboratory. Such proteins are typically expressed on the surface of a bacteriophage, and a functional property of the protein (most often binding to its biological target) is interrogated under increasingly harsh conditions. This technique is known as phage display, a directed evolution technique.

The MEFD molecular switch, i.e., controlled activation of the catalytic cytotoxic activity of the ribonuclease Bn provides yet another substantial, specific and credible utility, and the following specific advantages over phage display and other existing directed evolution techniques, in that:
1) The entire selection takes place inside a living bacterium, and stabilizing mutations are sorted from destabilizing mutations in the most efficient and decisive manner possible—life or death of that bacterium, respectively. This property greatly increases the throughput of the assay (i.e. the number of variants which can be tested within a given time). Throughput is the main consideration for the screening methods described above.
2) The MEFD molecular switch is applicable to more types of proteins. It does not require the protein of interest to have a known, binding activity. In many cases, biologically important proteins do not bind ligands, or the ligands that they bind are not amenable to phage display (e.g., not available in sufficient quantity, or too unstable to survive the harsh binding conditions employed).
3) The MEFD molecular switch bypasses limitations of expressing proteins on the phage surface. Only small (<20,000 Dalton) proteins can be displayed. In addition, surface display relies on a complex cellular pathway, and, for reasons which are not well understood, many protein sequences and/or structures are not able to be targeted to the viral membrane.
4) The MEFD molecular switch can be “tuned” to select for proteins of a desired stability. Tuning is achieved by introducing known stabilizing or destabilizing mutations into the Bn domain, in order to make the switch optimally responsive to a target stability range.

### 3.0 BRIEF DESCRIPTION OF THE DRAWINGS

FIG. 1A is a schematic illustration of the insert domain of the insert protein GCN4 in an unfolded conformation.

FIG. 1B is a schematic illustration of the insert domain, of the insert protein GCN4 in an folded conformation.

FIG. 1C is a schematic illustration of a conformation of the target protein. Bn having a folded target domain in the shape of a wedge and having a surface loop.

FIG. 1D is a schematic illustration of a conformation of the target protein Bn having an unfolded target domain in the shape of a straight line and having a surface loop.

FIG. 1E is a schematic illustration of the BG fusion protein capable of existing in two mutually exclusive conformations.

FIG. 1F is a schematic illustration of the BG fusion protein capable of existing in two mutually exclusive conformations, in which an equilibrium state has been influenced by the binding of a ligand.

FIG. 2 is an illustration of the constituent proteins of the of the barnase-GCN4 (BG) fusion protein.

FIG. 3 is a graph showing the urea-induced denaturation of the barnase-GCN4 (BG) fusion protein (filled circles) and the urea-induced denaturation of barnase (open squares), as monitored by Ttp fluorescence maximum.

FIG. 4 shows the urea dependence of the apparent dissociation constant, for intermolecular complementation (Bn fragments 1-67 and 68-110).

FIG. 5 shows two graphs. FIG. 5(a) is a graph showing the DNA binding-induced unfolding of the Bn domain of barnase-GCN4 (BG) fusion protein as monitored by Trp fluorescence maximum. FIG. 5(b) is a graph showing the inhibition of RNase activity by DNA binding.

FIG. 6 shows the CD spectra of free Bn and the barnase-GCN4 (BG) fusion protein in the absence and presence of AP-1 DNA.

### 4. DETAILED DESCRIPTION OF THE INVENTION

The following detailed description illustrates the invention by way of example, not by way of limitation of the principles of the invention. This description will, clearly enable one skilled in the art to make and use the invention, and describes what the inventors presently believe is the best mode of carrying out the invention. It is to be understood that, this invention is not limited to the particular embodiments described, as such may, of course, vary.

### 4.10 Lexicon

It is to be understood that the terminology used herein is for the purpose of describing particular embodiments only, and is not intended to be limiting, since the scope of the present invention will be limited only by the appended claims.

As used herein and in the appended claims, the singular indefinite forms “a”, “an”, and the singular definite form “the” include plural referents unless the contest clearly dictates otherwise. Thus, for example, reference to “a domain” includes a plurality of such domains and reference to “an energy state” includes reference to one or more energy states and equivalents thereof known to those skilled in the art, and so forth.

### 4.1.1 Lexicon: Domain

As used herein, the term “domain” means the molecular structure of an entire protein molecule or the molecular structure of a part, portion, or region, of the molecular structure of a protein molecule, including a part, portion, or region of the protein molecule's surface or the protein molecule's interior. A domain may refer only to a distinction in a protein molecule's structure, such as for example, an alpha helix or a beta sheet. A domain may or may not have an associated biological function, such as a regulatory, receptor, signaling, active, catalytic, or other biological function. A domain may further be associated with a free energy, i.e., a thermodynamic state function that indicates the amount of energy that stabilizes the domain when the protein, or part thereof, with which the domain is associated is in a folded configuration. All of part of the free energy may be available for the domain to do biochemical work.

As used herein, the term “insert domain” also means a “binding domain” and/or “regulatory domain.”

As used herein, the term “target domain” also means a “catalytic domain” and/or a “cytotoxic domain.”

### 4.1.2 Lexicon: Surface Loop

As used herein, the term “surface loop” means a continuous length of a polypeptide chain whose constituent amino acids is in neither an alpha helical conformation or in a beta sheet conformation, and can contact at least five water molecules, as determined by the DSSP computer program of Wolfgang Kabsch and Chris Sander. The DSSP, a program which is well known in the art, defines secondary structure, geometrical features and solvent exposure of proteins, given atomic coordinates in Protein Data Bank format, which is also well known in the art. (W. Kabsch & C. Sander, “Dictionary of protein secondary structure: pattern recognition of hydrogen-bonded and geometrical figures”, Biopolymers 22, 2577-2637. (1983); See also, Centre for Molecular and Bimolecular Informatics, University of Nijmegen, Toernooiveld 1, P.O. Box 9010, 6500 GL Nijmegen, +31 (0)24-3653391. As used herein the term surface loop further comprises a “target domain” associated with a second quantity of free energy.

### 4.1.3 Lexicon: First and Second Surface Loop Amino Acids

As used herein:
1) an alpha carbon of a “first amino acid of the surface loop” defines the beginning of a surface loop; and,
2) an alpha carbon of a “second ammo acid of the surface loop” defines the end of a surface loop.

### 4.2 Proteins

In their simplest form, proteins are polypeptides, i.e., linear polymers of ammo acid monomers. However, the polymerization reaction which produces a polypeptide results in the loss of one molecule of water from each ammo acid. Consequently, a polypeptide is more rigorously defined as a polymer of amino acid residues. Natural protein molecules may contain as many as 20 different types of amino acid residues, each of which contains a distinctive side chain.

An amino acid is an organic molecule containing an ammo group (“—NH₂”) and a carboxylic acid group (“—COOH”). While there are many forms of ammo acids, all of the important amino acids found in living organisms are alpha-ammo acids. Alpha amino acids have their both their —COOH and —NH₂ groups attached to the same carbon atom, which is called the alpha carbon atom.

Thus, all of the important amino acids found in living organisms consist of an alpha carbon atom to which there is attached:

1) A hydrogen atom

2) An amino group (—NH₂)

3) A carboxyl group (—COOH).

4) One of 20 different “R” groups.

It is the structure of the R group that, distinguishes each amino acid structurally and determines its biochemical properties. Moreover, the structure, and biochemical properties of a protein are by the precise sequence of the amino acids in the polypeptide chains of which it is comprised. One end of every polypeptide, called the amino terminal or N-terminal, has a fee amino group (—NH₂). The other end, has a free carboxyl group (—COOH), and is called the carboxyl terminal or C-terminal.

The particular linear sequence of amino acid residues in the polypeptide chain comprising a protein defines the primary structure of that protein. However individual polypeptides and groups of polypeptides undergo spontaneous structural alteration and association into a number of recurring intermediate patterns such as, for example, helices, including alpha helices, and sheets, including beta sheets. These recurring intermediate polypeptide patterns are referred to as a protein's secondary structure. The spontaneous structural alteration and association of polypeptide chains into a secondary structure is determined by the sequence of amino acids in the polypeptide chains and by the ambient biochemical environment.

The helices, sheets, and other patterns of a protein's secondary structure additionally undergo a process of thermodynamically-preferred compound folding to produce a three-dimensional or tertiary structure of the protein. The fully folded conformation of the protein is maintained by relatively weak inter-atomic forces such as, for example, hydrogen bonding, hydrophobic interactions and charge-charge interactions. Covalent bonds between sulphur atoms may also participate in protein folding into a tertiary conformation by forming intra-molecular disulfide bridges in a single polypeptide chain, as well as by forming intermolecular disulfide bridges between separate polypeptide chains of a protein. This ability of polypeptide chains to fold into a great variety of structures, combined with the large number of amino acid sequences of a polypeptide chain that can be derived from the 20 common amino acids in proteins, confers on protein molecules their great range of biological activity.

The tertiary structure of a protein may contain a surface loop.

Protein folding occurs on a global level that endows the entire protein molecule with a three dimensional structure and surface topology. Protein folding also occurs at a local level at multiple sites upon and within a protein. Locally, folding may involve one or more polypeptide subunits of the protein to endow different regions of the protein with different specific biological activities, or different specific molecular architectures, such as, for example, fashioning a location in a protein molecule into a receptor site for another molecule.

Because the folding of a protein molecule is both a global and local process, it can endow a protein molecule with both global, and local structural and biological properties, such as, for example, an enzymatic activity, or a capacity and specificity for binding other proteins, such as antigens. Consequently, the biological functions of a protein depend on both its global folded tertiary structure, which is also called its native or folded conformation, as well as the folded structure of regions of the protein. Conversely, a global, or local unfolding of a protein deactivates its global or local biological activity. An unfolded, biologically inactive protein is said to be in a denatured or unfolded conformation.

Many proteins are comprised of domains that, communicate with each other by means of conformational changes in the structure of the protein of which they are a part, in order to activate or deactivate a biological function. For example, in the case of a protein that is an enzyme, ligand binding or phosphorylation can serve as a switching mechanism to Induce structural changes within the enzyme's regulatory domain, which then triggers activity in the enzyme's catalytic domain.

Another type of switching mechanism is illustrated in vivo by proteins that are unfolded in physiological conditions but fold upon binding to a cellular target. In this molecular switching mechanism, the folding and unfolding of a regulatory domain of a protein modulates the function of the protein via propagation of structural changes to its active domain.

### 4.3 MEFD Molecular Switching Fusion Protein

The following preferred embodiment of the fusion protein of the present invention functions as a MEFD molecular switch and provides allosteric switching in molecular biology. The molecular mechanism of the mutually exclusive domain folding molecular switch:

1) is inherently cooperative; and,

2) behaves in a binary fashion; and,

3) is reversible; and,

4) is readily adjusted by external factors.

The fusion protein is synthesized from:
1) an insert protein having an insert domain lying between an amino terminal and a carboxyl terminal, which insert domain is associated with a first quantity of free energy; and,
2) a target protein having at least one surface loop that begins at an alpha carbon of a first amino acid of the surface loop and terminates at an alpha carbon of a second amino acid of the surface loop, which surface loop comprises a target domain associated, with a second quantity of free energy.

### 4.4 N—C Terminal Length

The amino terminal of the insert protein is spatially separated from the carboxyl terminal of the insert protein by a linear (i.e., straight line) distance known as the amino-carboxyl length (hereinafter, the “N—C terminal length”) of the insert protein, that is measured when the insert protein is in its folded con formation.

### 4.5 C_{α}-C_{α} Length

The alpha carbon of the first amino acid of the surface loop of the target protein is spatially separated from the alpha carbon of the second ammo acid of the surface loop of the target protein by a linear (i.e., straight line) distance known as the alpha-carbon-alpha-carbon length of the surface loop of the target protein (hereinafter, the “C_{α}-C_{α} length”), that is also measured when the target protein is in its folded conformation.

### 4.6 MEFD Topological Design Criterion

The molecular structure of the fusion protein is engineered so that, at any time, the folding of the insert domain necessarily unfolds the target domain, and vice versa, thereby making the folded and unfolded states of the insert and target domains mutually exclusive. This mutual exclusion of concurrently folded or concurrently unfolded, states is accomplished, by the insertion of the insert protein into the surface loop of the target protein subject to the topological criterion, wherein the N—C terminal length of the insert protein is at least two-times greater than the Cα-Cα length of the surface loop of the target protein.

The fusion protein of the present invention comprises a two-domain, bifunctional fusion protein, wherein the free energy released by the folding of a first domain of the fusion protein drives unfolding of a second domain of the fusion protein, and vice versa.

Subject to the topological design criterion, a dynamic state of thermodynamic and structural equilibrium is established in the fusion protein that disenables the insert domain of the insert protein and the target domain of the target protein from simultaneously co-existing in their native folded states.

Accordingly, any excess free energy present in one of the two domains that is not necessary to stabilize its folded configuration is spontaneously transferred, through the structure of the fusion protein, to the other of the two domains to unfold it from its folded configuration, and vice versa. In effect, the excess free energy stored, in the folded conformation of one domain is used to drive the unfolding of the other domain; and, the molecular structure of the fusion protein is engineered to create a dynamic state of thermodynamic and correlative structural equilibrium, that is determined by the relative thermodynamic and structural stabilities of the two domains.

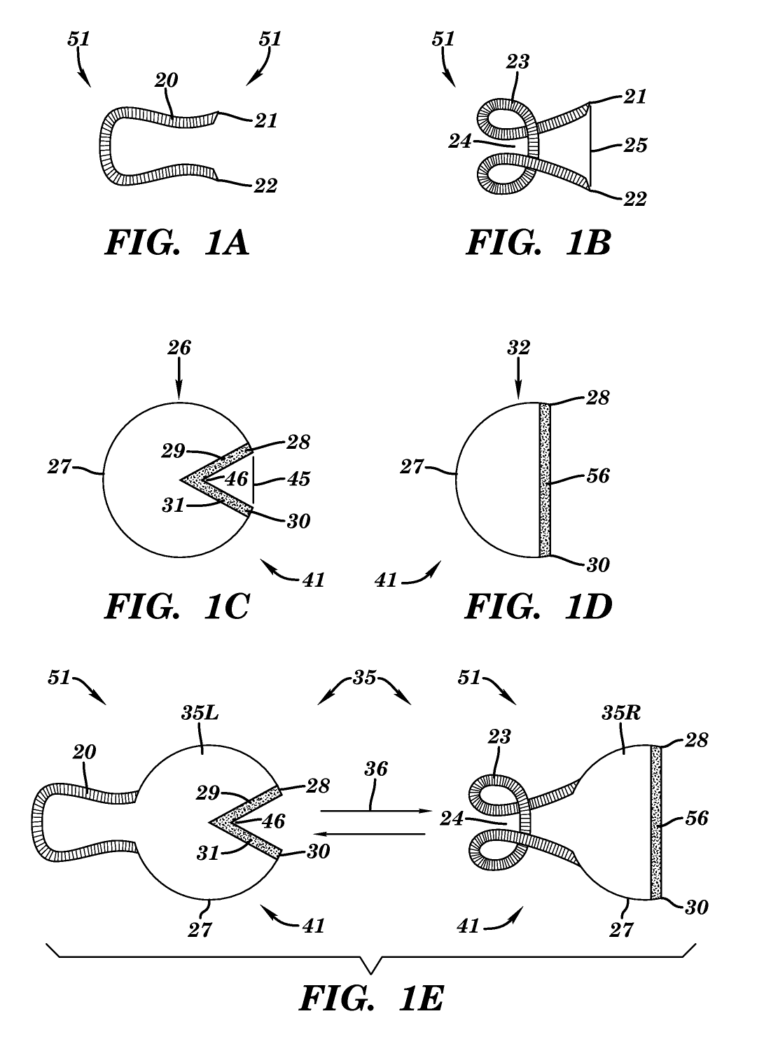

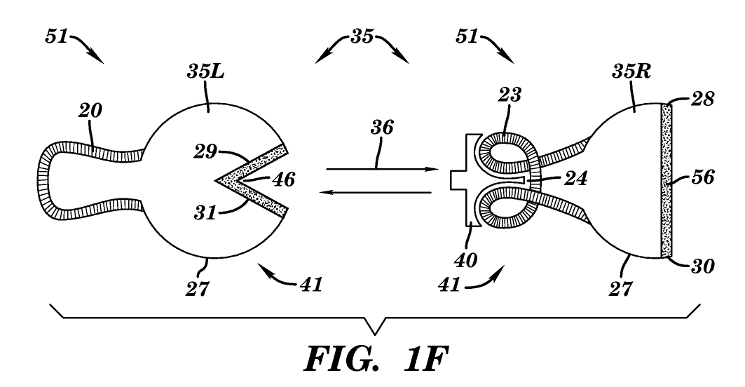

Viewed another way, the molecular structure of the fusion protein is engineered to create a MEFD molecular switch by creating cooperatively folding-unfolding subunits comprising two protein domains, which two domains cannot simultaneously exist in their folded states. This scheme is depicted in FIGS. 1A-F.

FIG. 1A shows a schematic illustration of the insert domain of the GCN4 insert protein in an unfolded conformation, in FIG. 1A, the GCN4 insert protein 51, having an amino terminal 21 and a carboxyl terminal 22, exists in an unfolded conformation 20, thereby forming an unfolded insert domain, schematically illustrated as a hatched ribbon that is coincident with the extent of the GCN4 insert protein 51.

FIG. 1B shows a schematic illustration of the insert domain of the GCN4 insert protein in an folded conformation. In FIG. 1B, the GCN4 insert protein 51, having an amino terminal 21 and a carboxyl terminal 22, exists in a folded conformation 23, thereby forming a folded insert domain, schematically illustrated as a hatched double-crossed ribbon that is coincident with the extent of the GCN4 insert protein 51, and folds to form indentation 24.

In FIG. 1B, reference numeral 25 refers to the amino-carboxyl length of the GCN4 insert domain in its folded conformation, which is synonymous with the N—C terminal length of the GCN4 insert domain in a folded conformation.

In FIG. 1C, there is shown schematically a folded conformation 26 of the Bn target protein 41 having an folded target domain in the shape of a wedge 46. Bn target protein 41 also has a surface loop 27, schematically shown as a nearly full circle, arising from an alpha carbon of a first amino acid 28 of the surface loop 27 of a first arm 29 of wedge 46, and ending at an alpha carbon of a second amino acid 30 of the surface loop 27 of a second arm 31 of wedge 46. Also shown schematically in FIG. 1C is line 45, representing the (straight) Cα-Cα length of the surface loop 27.

In FIG. 1D, there is shown schematically an unfolded conformation 32 of the Bn target protein 41 in which folded target domain 46 (of FIG. 1C) has unfolded into the shape of straight line 56. Unfolded conformation 32 of Bn target protein 41 also has surface loop 27, now shown, as a half-circle arising from the alpha carbon of the first amino acid 28 of the surface loop 27 and ending at the alpha carbon of the second amino acid 30 of the surface loop 27.

In FIG. 1E, there is shown schematically the BG fusion protein 35 including GCN4 insert protein 51 inserted into surface loop 27 of Bn target protein 41, which BG fusion protein 35 is capable of existing in two mutually exclusive conformations 35L and 35R, representing the mutually exclusive binary states of the MEFD molecular switch embodied in the BG fusion protein 35.

The image to the left of the antiparallel arrows 36 of FIG. 1E shows exclusive state 35L of BG fusion protein 35, wherein the GCN4 insert protein 51, with its insert domain in an unfolded (hatched ribbon) conformation 20, (as shown in FIG. 1A), has been inserted into surface loop 27 of the Bn target protein 41 with its target domain in its folded conformation 46, (as shown In FIG. 1C).

The image to the right of the antiparallel arrows 36 of FIG. 1E shows exclusive state 35R of the BG fusion protein 35, wherein GCN4 insert protein 51, with its insert domain in its folded (hatched double-crossed ribbon) conformation 23, (as shown in FIG. 1B), inserted into surface loop 27 of the Bn target protein 41 with its target domain in its unfolded (straight line) conformation 56 (as shown in FIG. 1D).

In FIG. 1F, BG fusion protein 35 is again shown schematically existing in two mutually exclusive conformations 35L and 35R, representing the mutually exclusive binary states of the MEFD molecular switch embodied in the BG fusion protein 35. However, the dynamic equilibrium existing between conformations 35L and 35R has been shifted to the right by the binding of the APT ligand 40 to the indentation 24 of insert domain of the GCN4 insert protein 51 in folded conformation 23.

If the insert domain of the GCN4 insert protein 51 in its folded conformation 23 (FIG. 1B, FIG. 1E Right and FIG. 1F Right) is more stable than the target domain (shown as having wedge 46 in FIG. 1C, FIG. 1E Left, and FIG. 1F Left) of the Bn target protein 41 in its folded conformation 26, (FIG. 1C), then the insert domain of the GCN4 insert protein 51 in its folded conformation 23 (FIG. 1B, FIG. 1E Right and FIG. 1F Right) will, have an excess of tree energy with which to forcibly stretch and untold the folded conformation 26 (FIG. 1C) of the Bn target, domain (shown as having wedge 46 FIG. 1C, FIG. 1E Left, and FIG. 1F Left) of the Bn target protein 41 (FIG. 1C, FIG. 1E Left and FIG. 1F Left), thereby unfolding wedge 46 into line 56, and yielding the BG fusion protein 35 in state 35R.

If target domain of target protein 41 in folded conformation 26 (FIG. 1C) is more stable than insert domain of insert protein 51 in folded, conformation 23 (FIG. 1B, FIG. 1E Right and FIG. 1F Right), then target domain (shown as having wedge 46 FIG. 1C, FIG. 1E Left, and FIG. 1F Left) of target protein 41 in its folded conformation 26 (FIG. 1C) will, have an excess of free energy with which to forcibly stretch and unfold insert domain of insert protein 51 in folded conformation 23 (FIG. 1B, FIG. 1E Right and FIG. 1F Right), thereby folding line 56 into wedge 46, and yielding fusion protein 35 in state 35L.

In this manner, the MEFD molecular switch fully exploits the free energy stored in the folded conformations of the aforementioned domains, as well as the inherent cooperatively of reciprocal domain folding, to create a molecular switch of unprecedented efficiency. Consequently, the MEFD molecular switch is a novel and powerful approach to understanding the fundamental mechanisms of allosteric switching in molecular biology and for the developing diagnostic and therapeutic proteins with novel capabilities, possessing the following advantages:
1) the mechanism of the molecular switch it is inherently cooperative; and,
2) the all-or-nothing action, of the mechanism of the molecular switch assures that it behaves in a binary fashion; and,
3) the switching mechanism is reversible; and,
4) the position of the folding/unfolding equilibrium can be readily adjusted, by external factors.

While the MEFD switch entails the creation of a two-domain, bifunctional fusion protein to be described more fully hereinafter, the MEFD switch disclosed herein is not limited, to the insertion of an insert protein into a target protein having only one domain or only one biological function. The MEFD switch disclosed herein comprises cases wherein one or more insert proteins is inserted into one or more surface loops of target proteins having multiple domains and multiple biological functions, the effect of these insertions being to form a one or more cooperatively folding-unfolding subunits in the resultant fusion protein, each comprising two protein domains, which two domains cannot simultaneously exist in their folded states, thereby forming one or more cooperative, reversible, MEFD molecular switches in the same fusion protein, each of which is responsive to different controllable effector signals such as, for example, ligand binding, pH, temperature, chemical denaturants, or the presence of stabilizing or destabilizing mutations in either the Bn or GCN4 domains.

The novel fusion protein herein, synthesized in accordance with the foregoing principles is a Barnase-GCN4 fusion protein (“BG”) comprising an insert protein, the ligand-binding polypeptide GCN4, (“GCN4”), having an insert domain, fused to a target protein, barnase (“Bn”) having a target domain, such that the aforementioned topological design criterion prevents GCN4 and Bn from existing simultaneously in their folded states. Their respective domains engage in a thermodynamic tug-of-war in which the more stable domain forces the less stable domain to unfold. In the absence of the ligand AP-1 consensus DNA oligonucleotide (“AP-1”), Bn is more stable than GCN4, and is therefore folded and active. In the presence of the AP-1, Bn is less stable than GCN4, and is therefore unfolded and inactive. The insert domain of GCN4 is substantially unstructured, infra.

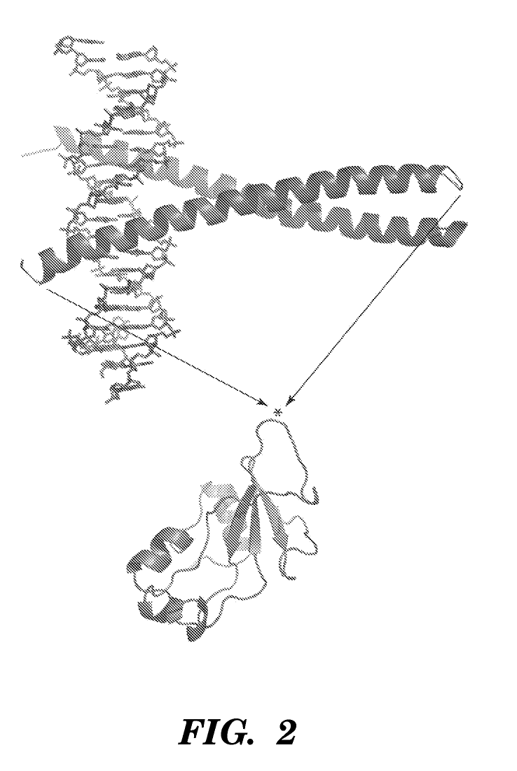

FIG. 2 is an illustration of the constituent proteins of the of the barnase-GCN4 fusion protein.

GCN4 is shown in the upper portion of FIG. 2 and barnase is shown in the lower portion of FIG. 2. The DNA binding region of GCN4 is represented by the left aspect of the thick transverse and horizontally oriented helices in the upper portion of FIG. 2. The coiled-coil region of GCN4 is represented by the right aspect of the thick transverse and horizontally oriented helices in the upper portion of FIG. 2. The DNA oligonucleotide bound to GCN4 is represented by the vertically-oriented double-helical structure in the upper right aspect of FIG. 2. The asterisk in the lower portion of FIG. 2 indicates the point at which GCN4 was inserted between amino acid residues 66 and 67 of a surface loop of barnase.

The GCN4 protein is a transcription factor that binds to the promoter element TGACTC and regulates a large number of yeast genes including genes encoding enzymes of amino acid biosynthetic pathways. Starvation of yeast cells for any of a number of amino acids leads to enhanced GCN4 protein synthesis through stimulation of GCN4 mRNA translation. Accordingly, GCN4 is the primary regulator of the transcriptional response to amino acid starvation.

Barnase is a bacterial protein that consists of 110 amino acids and has ribonuclease activity. It is synthesized and secreted by the bacterium. Bacillus amyloliquefaciens, but is lethal to the cell when expressed without its inhibitor barstar, The inhibitor binds to and occludes the ribonuclease active site, preventing barnase from damaging the cell's RNA after it has been synthesized but before it has been secreted.

AP-1 is a protein comprising a complex mixture of fun family (c-Jun, JunB, and JunD), homodimers and heterodimers with the Fos family (c-Fos, FosB, Fra-1, and Fra-2), or with Fos-related proteins, CREB or ATF-2.5. Its dimerization is mediated by a carboxy-terminal coil structure (motif), known as a leucine zipper, and is necessary for DNA binding to a palindromic sequence known as the TPA-responsive element (TRE) or AP-1 consensus site, existing in many gene enhancers.

AP-1 regulates gene expression either positively or negatively, depending on the interaction with different Fos/Jun or Jun/Jun dimers. Domain mapping experiments indicate that c-Jun interacts with the conserved C-terminus of TATA-binding protein and TFIIB in vitro. The AP-1 transcriptional complex has been implicated in a number of biological processes like cell cycle progression, differentiation, and transformation, c-Jun has also been linked to apoptosis.

BG binding to AP-1 induces folding of GCN4, forcibly unfolding and inactivating Bn. BG is thus a “natively unfolded” fusion protein that uses ligand binding to AP-1 to switch between partially folded conformations. The characteristic catalytic efficiency of Bn and the characteristic DNA binding affinity and sequence specificity of GCN4 are retained in the BG.

As indicated, supra, the constituent insert protein of BG comprises GCN4 which, in the lexicon of the instant patent application, is also called an insert, binding or regulatory domain. The insert domain of GCN4 lies between an ammo terminal and a carboxyl terminal and is associated with a first quantity of free energy. GCN4 has a 56 amino acid residue insert domain and functions biologically as a signaling marker or flag.

As indicated supra., the constituent target protein of BG is Bn, a 110 ammo acid residue ribonuclease produced exclusively by the bacterium Bacillus amyloliquefaciens. Bn has a surface loop that begins at an alpha carbon of a first amino acid of the surface loop and terminates at an alpha carbon of a second amino acid of the surface loop. The surface loop comprises a target domain of Bn. This target, domain is associated with a second quantity of free energy. When activated, the insert or catalytic domain of Bn is cytotoxic to all mammalian cell types.

In the absence of AP-1 binding, GCN4 can still dimerize via its C-terminal coiled-coil region with a dissociation constant (K_{d}) of 6-9 nM. The 25 N-terminal residues that comprise the DNA binding region of GCN4 are largely unstructured. The tact the DNA binding region of GCN4 is unstructured ensures that the barnase domain will be folded in the absence of DNA. An unstructured polypeptide is very flexible and can adopt any conformation. It can easily accommodate the folded barnase structure. When the 25 N-terminal residues that comprise the DNA binding region of GCN4 bind DNA, they essentially turn into a stiff rod, which is then incompatible with the folded barnase structure. Accordingly, The 25 N-terminal residues that comprise the DNA binding region of GCN4 uncouple folding/unfolding of the Bn domain with the coiled-coil region of GCN4 by acting as a long, flexible linker. Bn is consequently folded and active if no DNA is present.

The MEFD molecular switch embodies a novel molecular mechanism for regulating enzymatic activity. An insert domain of GCN4 in the present invention is Inserted into a target domain of barnase, as described in the 10/802,516 Application. The resulting fusion protein has a new function not present in either parent protein alone—it senses the presence of a specific DMA sequence, i.e., APT, and the enzymatic activity of barnase is switched on or off depending on whether that DNA sequence is absent or present.

The conformational equilibrium established between the insert protein, GCN4, and the target protein Bn is specifically reversible and controllable by means of ligand binding to AP-1.

### 4.7 Method

The GCN4 barnase fusion gene is made by first adding five amino acid linker (Gly-Thr-Gly-Ala-Ser) between the Lys66 and Ser67 codons of the barnase gene. The inserted DNA contains KpnI and NheI restriction sites that are used to introduce the ubiquitin gene.

KpnI and NheI restriction sites were created to fuse the Bn and GCN4 genes. The extra nucleotides introduced Gly-Thr and Ala-Ser at the junction points. These dipeptides serve as short linkers. GCN4 was inserted between residues 66 and 67 of the surface loop of Bn to create GB. The Cα-Cα distance between the ends of the surface loop is approximately 10 angstroms (A°).

The amino acids of the linker individually serve as short, flexible linkers at the points of attachment. The GCN4 gene is inserted between the Thr and Gly codons of the linker.

All genes are fully sequenced to verify their integrity.

An interim GCN4-barnase fusion expression plasmid pETMT is created by using NdeI and XhoI enzymes to insert the GCN4-barnase fusion gene into a plasmid, such as, for example, a pET25b(+) plasmid (Novagen), or any other T7 promoter-containing plasmid that also confers resistance to an antibiotic other than ampicillin.

The N—C terminal length of GCN4 of about 75 A°, ensures that DNA binding to BG will split the Bn insert domain, in two, thereby inactivating it.

In order to make the plasmid stable in II coli, the gene for bars tar, the intracellular inhibitor of barnase that is co-expressed with barnase by Bacillus amyloliquefaciens (together with its natural promoter from Bacillus amyloliquefaciens), is cleaved out of an pMT1002 plasmid (gift of Dr. Y. Bai, National Institutes of Health), or any other 17 promoter-containing plasmid that also confers resistance to an antibiotic other than ampicillin, with Gal and PstI enzymes. The barstar gene is then placed between Clal and PstI restriction sites on the pETMT plasmid (prior to this step, these sites are introduced using the QuikChange mutagenesis kit (Strategene)).

In order to obtain milligram quantities of the GCN4-barnase fusion protein, it is necessary to increase cellular levels of barstar and purify the inactive GCN4-barnase fusion-barstar complex. Accordingly, the barstar gene is cloned into a pET41 plasmid (Novagen), thereby placing it under control of a T7 promoter and conferring upon the transformed cells resistance to kanamycin or any other antibiotic other than ampicillin.

E. coli BL21 (DE3) cells are transformed with both plasmids, grown in a temperature range between about 20 degrees C. and 37 degrees C. in Luria-Bertani medium containing ampicillin and kanamycin to OD600=1.0, and induced with 100 mg/L IPTG. Bacteria are harvested about 2 to 12 hours later by centrifugation.

Cells are lysed in about 10 mM sodium phosphate (pH 17.5) by repeated freeze-thaw cycles in the presence of a small amount of lysozyme at a concentration of about lysozyme is 10 mg/liter. DNase I (Sigma) at a concentration of about 10 mg/liter is then added to reduce viscosity, and the solution is centrifuged to remove insolubles. 8 M urea is added to the supernatant to dissociate bound barstar, which is subsequently removed by passing the solution through DE52 resin (Whatman) or a substantially equivalent anion exchange chromatography resin. The solution is then loaded onto an SP-Sepharose column (Amersham-Pharmacia) or substantially equivalent cation exchange column, washed with 10 mM sodium phosphate (pH 7.5) and 6 M urea, and eluted with a 0-0.2 M NaCl gradient.

Western blot analysis using anti-GCN4 antibodies is used to show that the major impurities are truncated GCN4-barnase fusion, protein products in which the GCN4 domain, which is unfolded in the GCN4-barnase fusion protein-barstar complex, is partially digested. These proteins, however, elate significantly later than the intact GCN4-barnase fusion protein in the NaCl gradient. The urea is removed by dialysis against double-distilled water, to yield barnase-GCN4 fusion protein that is approximately 95% pure as judged by sodium dodecyl sulfate polyacryl amide gel electrophoresis.

### 4.8 Properties

The inventors herein characterized the structure and stability of BG by Tryptophan (“Trp”) fluorescence spectroscopy. Trp is an amino acid, that is naturally fluorescent. Three Trp residues are exclusively present in the amino acid sequence of Bn. In Trp fluorescence spectroscopy, Trp is illuminated with ultraviolet light (having a wavelength of about 280 nm) and it emits light of a longer wavelength. The wavelength of the emitted light depends on the molecular environment, around Trp. Free Trp emits at about 355 nm, which is wavelength that is emitted by Trp as part of the barnase domain in its unfolded conformation. This occurs because the local environment of Trp in unfolded barnase is comprised of water, which is also the case for free Trp, On the other hand, the emission wavelength of Trp in folded barnase is about 335 nm, because Trp is now surrounded by other hydrophobic amino acids. Because the three Trp residues are present only in the Bn sequence, Trp fluorescence reports primarily on the structure of the Bn domain and not the GCN4 domain.

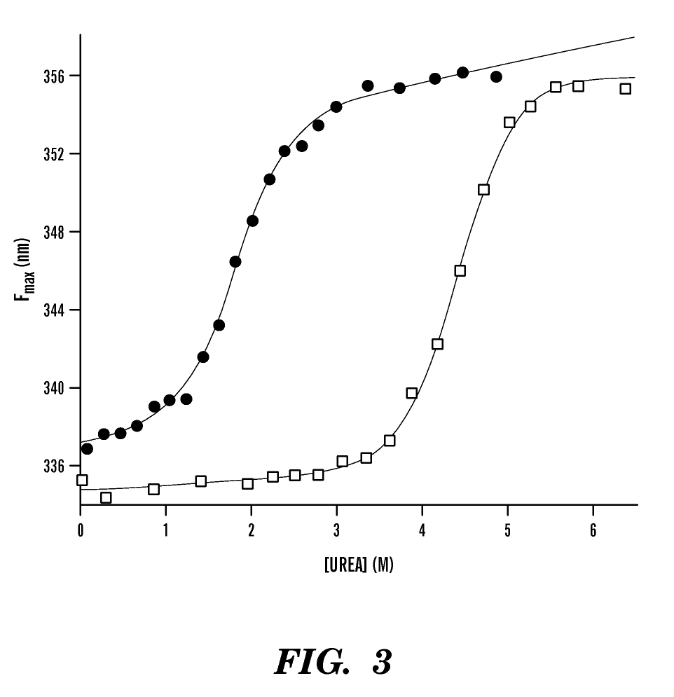

FIG. 3 is a graph showing the urea-induced denaturation of BG (filled circles) and the urea-induced denaturation of Bn (open squares), as monitored by Trp fluorescence maximum. In FIG. 3, the ordinate, showing the Trp fluorescence maximum in nanometers, is labeled Fₘₐₓ(nm), and runs from just below 336 nm to just above 356 nm in units of 4 nm; and, the abscissa, showing the molar concentration of urea, is labeled [Urea] (M), and is scaled in units of 1 M.

The graphs in FIG. 3 represent a best fit of the data to a linear extrapolation equation of the form

ΔG=ΔG(H₂O)−m*[denaturant] [equation 1]

where ΔG is the stability of the protein at a given denaturant concentration, ΔG(H₂O) is the stability of the protein in the absence of denaturant, m is a proportionality constant that depends on the protein, and [denaturant] is the concentration of denaturant in moles per liter.

Solution conditions are 200 nM protein (monomer concentration), 25 mM Hepes (pH 7.0), 100 mM NaCl at 25 8C. Data were collected on a Fluoromax-3 fluorometer (Jobin-Yvon/SPEX) with an excitation wavelength, of 280 nm. Emission maxima were calculated using the Datamax software package (Jobin-Yvon/SPEX). BG was expressed in Escherichia coli BL21 (DE3) and purified using the same protocol developed for barnase-ubiquitin fusion protein disclosed in the 10/802,516 Application. However, unlike the barnase-ubiquitin fusion protein, BG is found completely in inclusion bodies and is thus protected from proteolysis. The yield of BG is correspondingly much higher than that of barnase-ubiquitin.

As shown in FIG. 3, free Bn exhibits fluorescence emission maxima (Fₘₐₓ) of 335 nm and 356 nm in native and unfolded states (6 M urea), respectively. Fmax of BG is 337 nm in the absence of denaturant, suggesting that the Bn domain is folded in phi 7 buffer. Addition of urea unfolds both free Bn and the Bn domain of BG in a cooperative and reversible manner. Fitting these data to a linear extrapolation equation as described in Santoro, M. M. & Bolen, D. W. (1988). Unfolding free energy changes determined by the linear extrapolation method. 1. Unfolding of phenylmethanesulfonyl alpha chymotrypsin using different denaturants. Biochemistry, 27, 8063-8068, yields folding free energies of 7.4 kcal mol⁻¹ and 3.7 kcal mol⁻¹, respectively. It is apparent that insertion of the GCN4 domain, when it is largely disordered in the absence of DNA, does not unfold Bn.

To assure that DNA binding to the GCN4 domain of BG unfolds the Bn domain, the inventors monitored the Fₘₐₓ of BG as a function of AP-1 concentration. Fₘₐₓ measures the relative amount of folded v. unfolded Bn. To accomplish this it was first necessary to establish conditions that minimize intermolecular complementation of the Bn fragments that are generated in the course of GCN4 domain-DNA binding and folding. Intermolecular complementation is a direct consequence of the mutually exclusive folding mechanism. It occurs when the N-terminal Bn fragment binds with the C-terminal Bn fragment from another molecule.

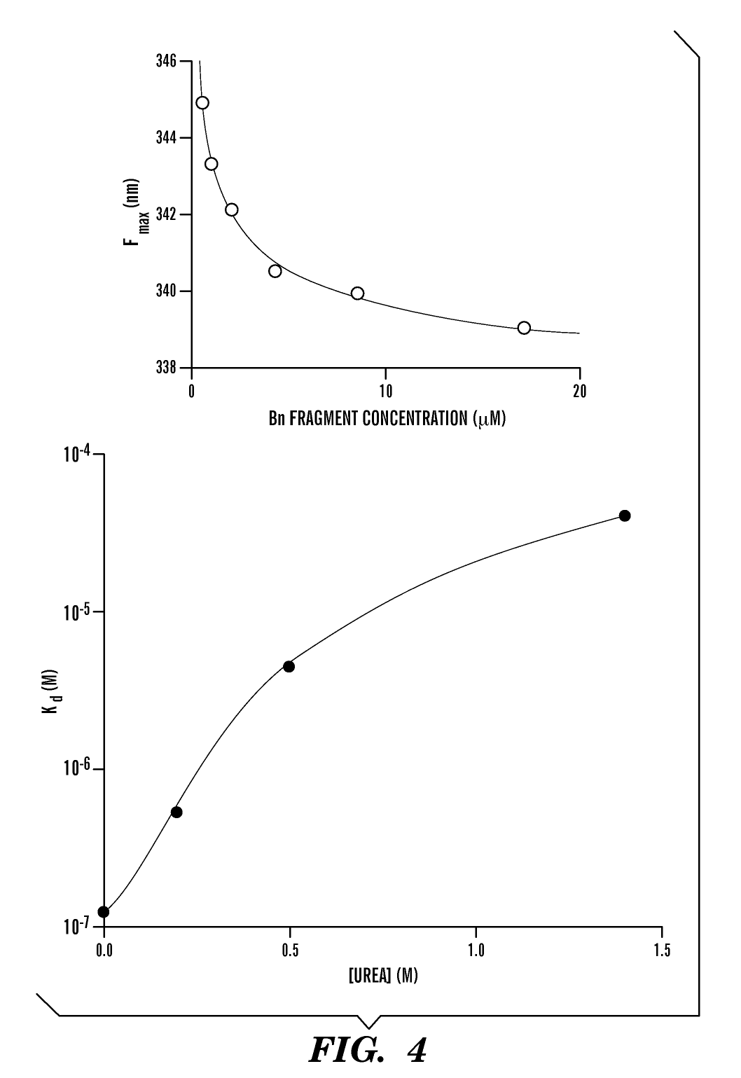

FIG. 4 is a graph showing the urea dependence of the apparent dissociation constant for intermolecular complementation (Bn fragments 1-67 and 68-110). In FIG. 4, the ordinate, showing the apparent dissociation constant, is labeled K_{d}(M), and is scaled in units of 10^{-1×n}; and, the abscissa, showing the molar concentration of urea, is labeled j Urea) (M), and is scaled in units of 0.5 M. The graph, is meant to guide the eye only. Various concentrations of 1-67 and 68-110 Bn fragments, always present at a 1:1 ratio, were unfolded in 6 M urea then rapidly diluted to the urea concentration indicated. Refolding of the complex was monitored by shift in Trp fluorescence maximum as shown in the inset, for which data were obtained in 0.2 M urea. K_{d} values were obtained, by fitting fluorescence maxima to a simple 1:1 binding equation; the continuous line in the inset indicates curve fit. Solution conditions are the same for FIG. 3. Bn fragments 1-67 and 68-110 were prepared by digesting purified S67M mutant with CNBr, according to the method of Matsudaira, P. (1990). Limited N-terminal sequence analysis. Methods Enzymol. 182, 602-613.

As shown in FIG. 4, the resulting complex can refold to a species that exhibits native-like fluorescence spectra.

To determine the apparent K_{d} for complementation, the inventors herein dissolved various concentrations of Bn fragments 1-67 and 68-110 in 6 M urea and refolded them by dilution into buffer. Formation of the native complex was monitored by a shift in Fmax. The data are well fit by the simple 1:1 binding equation, shown in the inset, of FIG. 4, which yields an apparent K_{d} value for complementation of about 100 nM (FIG. 4).

As expected, binding weakens with increasing urea concentration, reflecting the coupling between binding and folding. The inventors herein chose to perform the DNA binding experiments in 1.4 M urea because it disrupts intermolecular complementation while allowing the Bn domain of BG to remain largely folded, as shown in FIG. 3.

Destabilizing Bn by mutation should in principle produce a similar effect and eliminate the need for urea.

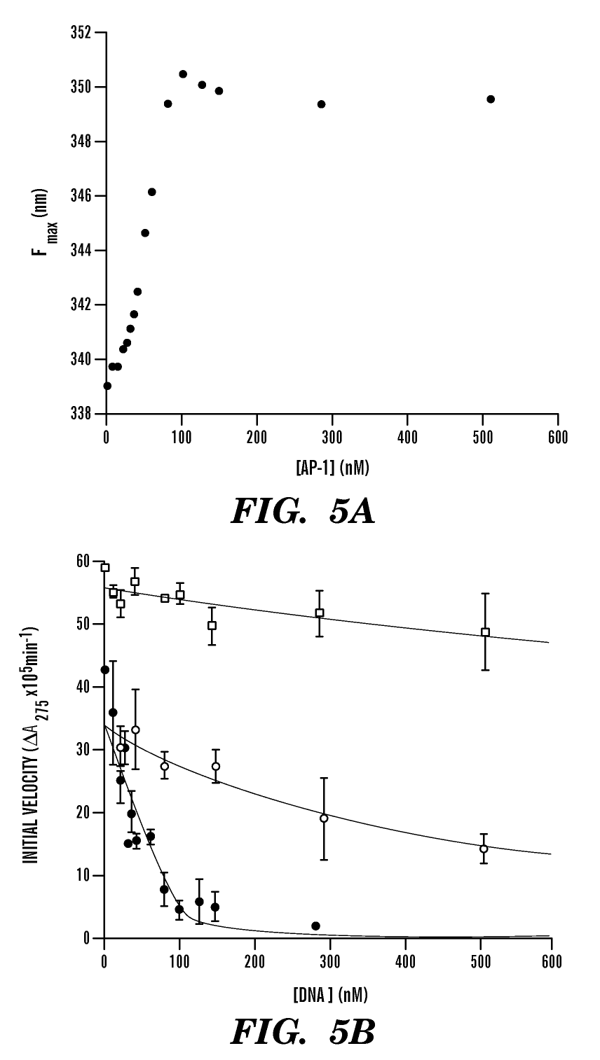

As shown in FIG. 5(a), AP-1 binding by the GCN4 domain induces a large shift in fluorescence of the Bn domain.

FIG. 5(a) is a graph showing the DNA binding-induced unfolding of the Bn domain of the barnase-GCN4 fusion protein as monitored by Trp fluorescence maximum. In FIG. 5(a), the ordinate, showing the Trp fluorescence maximum in nanometers, is labeled Fₘₐₓ(nm), and runs from 338 nm to 352 nm in units of 2 nm; and the abscissa, showing the nanomolar concentration of AP-1, is labeled [AP-1] (nM) and runs for 0 to 600 in units of 100 nM. In FIG. 5(a), solid circles designate the barnase-GCN4 fusion protein Incubated with AP-1.

To obtain the graph shown in FIG. 5(a) samples were incubated with for 2 h with the AP-1 oligonucleotide (5′-AGTGGAGATGACTCATCTCGTGC-3′) prior to measurements

The Fmax value of the fully bound species is 350 nm. The Fmax value of the urea unfolded state of BG extrapolates to 353 nm at 1.4M urea, as shown in FIG. 3, suggesting that DNA binding induces nearly complete unfolding of the Bn domain. The binding curve in FIG. 5(a) is linear and breaks sharply at the point where the AP-1 concentration equals that of the BG dimmer, i.e., at 100 nM. DNA binding is therefore stoichiometric and K_{d} is too low to be determined accurately. The inventors estimate the upper limit of K_{d} to be about 5 nM. This value is consistent with the minimal mechanism of scheme 1:

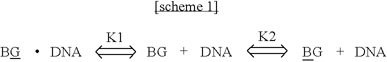

In scheme 1:

BG is the dimeric coiled-coil form of the fusion protein;

the presence of underscoring indicates that the domain is folded; and,

the absence of underscoring indicates that the domain is unfolded.

The observed K_{d} for DNA binding is equal to K₁(1+K₂), where K₁ is the intrinsic dissociation constant for the GCN4-DNA interaction in the absence of a structured Bn domain; and, K₂ is the equilibrium constant for Bn folding when the DNA binding region of GCN4 is unstructured. K₁ has been reported to be 2-20 nM for free GCN4. Cranz, S., Berger, C, Baici, A., Jelesarov, I. & Bosshard, H. R. (2004). Monomeric and dimeric bZIP transcription factor GCN4 bind at the same rate to their target DNA site. Biochemistry, 43, 718-727; Hollenbeck, J. J. & Oakley, M. G. (2000). GCN4 binds with high affinity to DNA sequences containing a single half-site. Biochemistry, 39, 6380-6389.

Extrapolation of the Gibbs free energy ΔG to 1.4 M urea yields K₂=4.2 (FIG. 3). The observed K_{d} value for the BG-DNA complex is thus predicted to be 1.0-100 nm, in reasonable agreement with FIG. 4. The higher than expected affinity of BG compared to free GCN4 may be due to the use of Hepes buffer in the inventor's experiments and phosphate buffer in the GCN4 studies reported in the preceding paragraph. Nevertheless, it is evident that BG retains the tight binding affinity of the parent. GCN4 protein.

To assure that DNA binding switches off enzymatic function, the inventors herein measured BG ribonuclease activity under the same conditions as those used, for FIG. 5(a).

FIG. 5(b) is a graph showing the inhibition of RNase activity by DNA binding. In FIG. 5(b), the ordinate, showing initial velocity, is labeled Initial Velocity (Δ₂₇₅×10⁵ min⁻¹), and runs front 0 to 60 in units of 10; and the abscissa, showing the nanomolar concentration of AP-1 or the non-consensus oligonucleotide 5′-CAGGGTGCTATGAACAAATGCCTCGAGCTGTTCCGT-3′, is labeled [DNA] (nM) and runs for 0 to 600 in units of 100 nM.

In FIG. 5(b):
solid circles designate the barnase-GCN4 fusion protein incubated with AP-1;
open circles designate the barnase-GCN4 fusion protein incubated with the non-consensus oligonucleotide 5′-CAGGGTGCTATGAACAAATGCCTCGAGCTGTTCCGT-3′; and,
open squares represent free Bn incubated with AP-1.

In FIG. 5(b), fitted K_{d} values are about 2 nM for filled circles; about 360 nM for open circles; and, about 3 μM for open squares. Error bars represent standard, deviations of three measurements. Samples were prepared as for FIG. 5(a) and were assayed for RNase activity by addition of 20 μM guanylyl(3′-5′)uridine 3′-monophosphate. Substrate transesterification was monitored by absorbance at 275 nm on a Gary 100 spectrophotometer (Varian Instruments). Initial velocities were obtained from least-squares tits of the linear portion of the data. The concentration of free Bn was reduced to 60 nM to lower the initial velocity to measurable levels. Conditions are identical to those for FIG. 3 except that 1.4 M urea is present in all samples, and samples for enzyme assays contain 0.1 mg ml 1 bovine serum albumin.

As shown in FIG. 5(b), one equivalent of AP-1 reduces activity to background levels. One explanation is that DNA acts as a competitive inhibitor by binding to the BG active site. But, as shown in FIG. 5(b), this possibility was eliminated by the finding that AP-1 has little effect on the activity of free Bn. Moreover, inhibition is sequence specific, and as further shown in FIG. 5(b), AP-1 inhibits BG≧100-fold more effectively than a non-consensus DNA oligonucleotide. The apparent K_{d} values for binding the consensus and non-consensus sequences are about 2 nM and 360 nM, respectively.

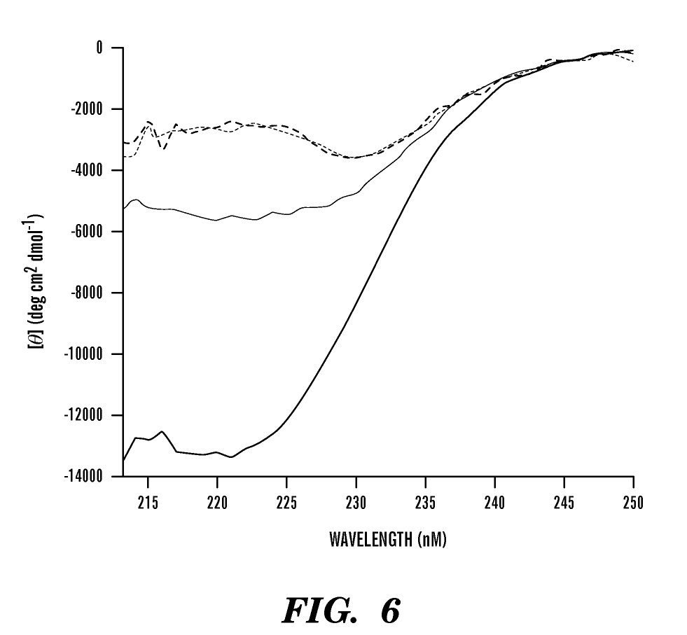

To further characterize the DNA-induced conformational transition, the inventors measured circular dichroism (CD) spectra of BG and free Bn in the presence and absence of AP-1. The results are shown in FIG. 6.

FIG. 6 shows the CD spectra of free Bn and BG in the absence and presence of AP-1 DNA. In FIG. 6, the CD spectrum of free Bn in the absence of AP-1 DNA is shown as a broken grey line and the CD spectrum BG in the absence of AP-1 DNA is shown, as a continuous grey line. In FIG. 6, the CD spectrum of free Bn in the presence of AP-1 DNA is shown as a broken black line and the CD spectrum BG in the presence of AP-1 DNA is shown as a continuous black line. Protein and AP-1 concentrations were 0.50 μM and 0.94 μM, respectively. The black-coded spectra were generated by subtracting the spectra of free AP-1 at the same concentration. Solution conditions are identical to those for FIG. 5. Data were collected on a model 202 spectropolarimeter (Aviv Biomedical, Inc.) in a 1 cm×1 cm cuvette. Wavelengths below 212 nm are not shown due to excessive sample absorbance.

The CD spectra of Bn are identical with and without AP-1, This result corroborates the enzyme assay results shown in FIG. 5(b), and confirms that Bn does not bind the DNA oligonucleotide.

Compared to Bn, BG displays enhanced ellipticity with a broad minimum near 222 nm in the absence of AP-1. This finding suggests that the GCN4 domain is partially helical and is consistent with the concept that BG exists as a coiled-coil dimer when no DNA is present. The presence of a partial, helical structure may be responsible for the lower enzymatic activity of BG relative to Bn (FIG. 5(b)).

In marked contrast to Bn, BG exhibits a large change in ellipticity at: 222 nm upon addition of AP-1. The change in molar ellipticity value ([Θ]₂₂₂) of −13,400 deg cm² dmol corresponds to a helix content of 32%, in close agreement with the predicted value of 33% if the GCN4 domain (56 residues of 170 total in BG) is fully helical. Taken together, FIG. 5(a), FIG. 5(b) and FIG. 6 demonstrate that:
1) enzymatic activity is regulated exclusively by DNA binding to the GCN4 domain;
2) binding is both tight and sequence-specific; and,
3) DNA binding unfolds the Bn domain.

The mutually exclusive mechanism is also proven by demonstrating that DNA binding affinity and Bn stability are coupled in an inverse fashion. Since APT binds stoichiometricaliy to BG (FIG. 4), a straightforward test comprises stabilizing the Bn domain, and determining whether K_{d} increases to a measurable value. At least a 20-fold increase in K_{d} would be required, corresponding to K₂≧100 and a change in the Gibbs free energy (ΔΔG)≧2.8 kcal mol⁻¹.

The inventors herein attempted to stabilize Bn by binding it to the mononucleotide inhibitor 3′-guanylic acid (3′-GMP) in 1.4 M urea. The 3′ GMP affinity is too low, however, to generate appreciable amounts of the complex at the highest nucleotide concentration permissible in the assay, about 200 μM; limited by excessive absorbance at 280 nm). Similarly, phosphate has been, shown to bind free Bn, but 50 mM phosphate stabilizes the Bn domain of BG by only 1.0 kcal mol⁻¹ under the conditions used, for FIG. 3 (data not shown). An alternate approach is to stabilize the Bn domain by introducing mutations. A Bn variant harboring substitutions at six positions has been found to be 3.0 kcal mol⁻¹ more stable than wild type. Serrano, L., Day, A. G. & Fersht, A. R. (1993). Stepwise mutation of barnase to binase: a procedure for engineering increased stability of proteins and an experimental analysis of the evolution of protein stability. J. Mol. Biol. 233, 305-312.

BG and its cousin, barnase-ubiquitin, serve as a platform for the design of enzymes that possess novel sensor capabilities. The main requirement is that the end-to-end length of the inserted protein must be longer than the distance between termini of the surface loop of the target protein. The ratio of these distances is about 7.5 for BG and about 4.0 for barnase-ubiquitin. The minimum value has not been determined. Another consideration is that, the stabilities of the two domains should be roughly comparable. If the catalytic domain is very stable, the affinity of the binding domain will be weakened and large concentrations of ligand will be required to trigger unfolding.

The inventors' experiments suggest that the optimal condition is when the catalytic domain is only marginally stable (e.g., ΔG=0.9 kcal mol⁻¹ in 1.4 M urea; FIG. 3), so that it is catalytically active in the absence of ligand but unfolded by low concentrations of ligand. The switching mechanism is highly responsive under these circumstances. Bn is a particularly useful catalytic domain because it otters the potential for therapeutic applications. It is highly toxic when introduced into both prokaryotic and mammalian cells. By linking Bn to an appropriate binding domain, the fusion protein, could have the ability to selectively kill, cell types depending on whether a specific ligand is present or not. Stabilizing or destabilizing mutations can be introduced into either domain to fine-tune the position and sensitivity of the conformational switch to match the needs of the application.

The foregoing description of the specific embodiments will so fully reveal the general nature of the Invention that others can, by applying knowledge within the skill of the art (including the contents of the references cited herein), readily modify and/or adapt for various applications such specific embodiments, without undue experimentation, without departing from the general concept of the present invention.

While this Invention has been described in connection with specific embodiments thereof, it will be understood that it is capable of further uses, variations modifications or adaptations. Such uses, variations, modifications and adaptations are intended to be within the meaning and range of equivalents of the disclosed embodiments, based on the teaching and guidance presented herein.

Having fully described this invention, it will also be appreciated by those skilled in the art that the same can be performed within a wide range of equivalent parameters, concentrations, and conditions without departing from the spirit and scope of the invention and without undue experimentation.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

It is believed that the disclosure set forth above encompasses multiple distinct inventions with independent utility. While each of these inventions has been disclosed in its preferred form, the specific embodiments thereof as disclosed and illustrated herein are not to be considered in a limiting-sense as numerous variations are possible.

The subject matter of the inventions includes all novel and non-obvious combinations and subcombinations of the various elements, features, functions and/or properties disclosed herein.

No single feature, function, element or property of the disclosed embodiments is essential to all of the disclosed inventions. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements and/or properties may be claimed through amendment of the present claims or presentation of new claims in this or a related application.

Such amended or new claims, whether they are directed to a different invention or directed to the same invention, whether different, broader, narrower or equal in scope to the original, claims, are also regarded as included within the subject matter of the inventions of the present disclosure.

We claim:

1. A fusion protein embodying a mutually exclusive folding domain molecular switch, the fusion protein comprising an insert protein having an insert domain lying between an amino terminal and a carboxyl terminal of the insert protein, the insert domain being associated with a first quantity of free energy; and,
a target protein having a surface loop that begins at an alpha carbon of a first amino acid of the surface loop and terminates at an alpha carbon of a second amino acid of the surface loop, the surface loop comprising a target domain of the target protein, the target domain being associated with a second quantity of free energy, wherein, the insert protein is inserted within the surface loop between the alpha carbon of the first amino acid of the surface loop and the alpha carbon of the second amino acid of the surface loop, such that an N—C length of the insert protein is at least two-times greater than a Cα-Cα length of the surface loop of the target protein.

2. The fusion protein of claim 1, wherein the insert domain exists in either a folded or unfolded conformation and the target domain exists in either a folded or unfolded conformation, the insert domain and the target domain comprising a cooperative and reversible conformational equilibrium such that if the Insert domain is in its folded conformation, the target domain is in its unfolded conformation and vice versa.

3. The fusion protein of claim 2, wherein the insert domain and the target domain are disenabled from simultaneously co-existing in their respective folded conformations.

4. The fusion protein of claim 2, wherein the insert domain and the target domain are disenabled from simultaneously co-existing in their respective unfolded conformations.

5. The fusion protein of claim 2, wherein any excess of the first quantity of free energy of the insert domain that is not necessary to stabilize the insert domain in its folded conformation is spontaneously transferred, through the structure of said fusion protein, to the target domain to unfold it from its folded conformation.

6. The fusion protein of claim 2, wherein any excess of the second quantity of free energy of the target domain that is not necessary to stabilize the target domain in its folded conformation is spontaneously transferred, through the structure of said fusion protein, to the insert domain to unfold it from its folded conformation.

7. The fusion protein of claim 2, wherein all or part of the first quantity of free energy is made available, to drive a folding of the target domain from its unfolded conformation by means of a controllable effector signal.

8. The fusion protein of claim 2, wherein the insert protein comprises GCN4, the insert domain comprises a regulatory or binding domain of GCN4, the target protein comprises barnase, the target domain comprises a catalytic or cytotoxic domain of barnase, the N—C length is about 75 Å, the first amino acid of the surface loop comprises proline in the number 64 position (“Pro64”), the second amino acid of the surface loop comprises threonine in the number 70 position (“Thr70”), and the Cα-Cα is about 10 Å.

9. The fusion protein of claim 2, wherein GCN4 is inserted between amine acid residues 66 and 67 of the surface loop of barnase.

10. The fusion protein of claim 2 wherein the regulatory or binding domain of GCN4 and the catalytic or cytotoxic domain of barnase comprise a cooperative and reversible conformational equilibrium, that may be determined by a controllable effector signal.

11. The fusion protein of claim 10, wherein the controllable effector signal comprises AP-1.

12. A method for the production of a GCN4-barnase fusion protein, comprising the steps of:
a. selecting a linker containing first and second restriction sites between a Lys66 and a Ser67 codon of a barnase gene;
b. using the first and second restriction sites of the linker to operationally insert a GCN4 gene between two amino-acid codons of the linker, thereby creating a GCN4-barnase fusion gene;
c. fully sequencing the GCN4-barnase fusion gene to verify its integrity;
d. using enzymes to operationally insert the GCN4-barnase fusion gene into any plasmid of a BL21 (DE3) family, thereby creating an interim GCN4-barnase fusion expression plasmid;
e. inserting a gene for barstar and its natural promoter from Bacillus amyloliquifaciens into the interim GCN4-barnase fusion expression plasmid, thereby creating a GCN4-barnase fusion-barstar complex plasmid;
f. cloning the gene for barstar into a T7 promoter-containing plasmid conferring resistance to an antibiotic other than ampicillin onto cells transformed by the T7 promoter-containing plasmid, thereby creating a barstar plasmid;
g. transforming E. coli BL21 (DE3) cells grown at about 20 to 37 degrees C in any medium compatible with E. coli growth using both the barstar plasmid and the GCN4-barnase fusion-barstar complex plasmid, and inducing the E. coli BL21 (DE3) cells with about 100 mg/L isopropyl b-D-thiogalactopyranoside;
h. harvesting the transformed E. coli cells by centrifugation after about 2 to 12 hours; after the induction;
i. placing the harvested E. coli cells in 10 mM sodium phosphate at a pH of 7.5, thereby creating a solution of harvested E. coli cells;
j. lysing the solution of harvested E. coli cells by repeated freeze-thaw cycles in the presence of about 10 mg/liter lysozyme, thereby creating a lysate;
k. adding about 10 mg/liter DNase I to reduce the viscosity of the lysate;
l. centrifuging the reduced viscosity lysate to remove insoluble, thereby forming a supernatant;
m. adding about 8 M urea to the supernatant to dissociate bound barstar;
n. removing the dissociated barstar from the supernatant by passing the supernatant through an anion exchange chromatography resin to yield a solution;
o. loading the solution onto a cation exchange column;
p. washing the solution with about 10 mM sodium phosphate (pH about 7.5) and about 6 M urea;
q. eluting the solution using a 0 to 0.2 M NaCl gradient;
r. removing the urea from the dilution by dialysis against double-distilled water to yield GCN4-barnase fusion protein.
